(19) United States
(12) Patent Application Publication
(10) Pub. No.: US 2026/0130636 A1
(43) Pub. Date: May 14, 2026

(54) IMAGE DISPLAY IN A LIMITED-ANGLE TOMOGRAPHY METHOD

(71) Applicant: Siemens Healthineers AG, Forchheim (DE)

(72) Inventors: Stephan Kellnberger, Zorneding (DE); Alexander Preuhs, Erlangen (DE)

(21) Appl. No.: 19/383,749

(22) Filed: Nov. 9, 2025

(30) Foreign Application Priority Data
Nov. 8, 2024 (DE) 10 2024 210 779.4

(51) Int. Cl. A61B 6/46; A61B 6/00; A61B 6/02; G06T 7/13; G06T 7/73; G06T 15/20

(52) U.S. Cl. CPC A61B 6/463; A61B 6/02; A61B 6/5223; G06T 7/13; G06T 7/73; G06T 15/205; G06T 2207/10081; G06T 2207/10116; G06T 2207/30004; G06T 2210/21

(57) ABSTRACT

For image display in a limited-angle tomography method, a plurality of projection images that depict an object are obtained, and based thereon, a three-dimensional image reconstruction is created. A first slice image for a first sectional plane through the object is created based on the image reconstruction, and a region of interest of the object in the first slice image is determined. In the first sectional plane, an intersection line of a second sectional plane that runs through the region of interest is determined. Depending on a position of the intersection line and/or depending on a geometric extent of the region of interest in the first slice image, a computation region around the second sectional plane is determined. Based on the plurality of projection images, a second slice image for the second sectional plane in accordance is created with the computation region.

This application claims the benefit of German Patent Application No. DE 10 2024 210 779.4, filed on Nov. 8, 2024, which is hereby incorporated by reference in its entirety.

### BACKGROUND

The present embodiments are related to image display in a limited-angle tomography method.

Limited-angle tomography (LAT) methods are x-ray imaging methods in which a stack of slice images may be created for a limited number of two-dimensional projection images. Limited-angle tomography methods will also be referred to here and below as tomosynthesis methods.

Tomosynthesis methods make possible a three-dimensional visualization of anatomical structures with lower radiation loads than, for example, in computed tomography CT or cone beam CT. The recording of a number of projection images from various angles around the patient (e.g., with various projection directions) enables a series of thin slice images to be created, which may be reconstructed into a three-dimensional image reconstruction, also referred to as a volume dataset. This makes possible a better visualization of complex structures, such as, for example, the lungs, and allows radiologists to identify and characterize lesions with a lower radiation dose.

In conventional tomosynthesis methods, the image quality obtained is as a rule not sufficient for carrying out an image-guided intervention (e.g., a bronchoscopy) with the methods. This restriction is attributable to the limited-angle acquisition and to the typically small number of projection directions, which may cause geometric distortions and stripe artifacts.

Approaches have been disclosed for compensating for the missing parts of the projection with the aid of deep learning algorithms, such as, for example, in Y. Huang et al., “Data consistent artifact reduction for limited-angle tomography with deep learning prior,” International workshop on machine learning for medical image reconstruction, Cham: Springer International Publishing, 2019. A disadvantage of this approach is the requirement for large amounts of annotated training data, which may be problematic precisely in the clinical context due to data protection regulations and the like.

As an alternative, it has been proposed by F. Saad et al., “Deformable 3/3D CT-to-digital-tomosynthesis image registration in image-guided bronchoscopy interventions,” Comput. Biol. Med. 171: 108199 that a registration and reconstruction method be used for improving the image quality of the tomosynthesis by diagnostic CT scans being used that have been recorded before the tomosynthesis scan. In this case, the previous CT image is reconciled with the intraoperative tomosynthesis image and used as a first estimation for the reconstruction. The additional CT scans increase the overall radiation dose applied.

### SUMMARY AND DESCRIPTION

The scope of the present invention is defined solely by the appended claims and is not affected to any degree by the statements within this summary

The present embodiments may obviate one or more of the drawbacks or limitations in the related art. For example, a possibility for displaying images in limited-angle tomography, by which the image quality is enhanced and the aforementioned disadvantages are overcome or reduced, is provided.

The present embodiments are based on the knowledge that, because of the principle of limited-angle data acquisition, artifacts are not equally strongly pronounced for all angles of view or slice planes through the imaged object. As a consequence, it is provided that a region of interest of the object be determined in a first sectional plane through the object, and a computation region for creation of a slice image be defined in accordance with a second sectional plane differing from the first sectional plane as a function of the region of interest defined in this way.

In accordance with one aspect of the present embodiments, a computer-implemented method for image display in a limited-angle tomography method is specified. In this method, a plurality of projection images that represents an object and a plurality of different projection directions is obtained, and based on the plurality of projection images, a three-dimensional image reconstruction is created. A first slice image for a first sectional plane (e.g., a predetermined first sectional plane) through the object is created based on the image reconstruction, and a region of interest of the object is determined in the first slice image. In the first sectional plane, an intersection line of a second sectional plane (e.g., of a predetermined second sectional plane) that intersects the first sectional plane and runs (e.g., in the first sectional plane) through the region of interest is determined. Depending on a position of the intersection line in the first sectional plane (e.g., the position of the intersection line relative to the region of interest) and/or depending on a geometric extent of the region of interest in the first slice image, a computation region about the second plane is determined. The computation region has a position and a slice thickness. Based on the plurality of projection images, a second slice image is created for the second sectional plane in accordance with the computation region and is displayed, for example (e.g., on a display device).

Unless specified otherwise, all acts of the computer-implemented method may be carried out by a data processing system that includes at least one data processing device. For example, the at least one data processing device is configured or adapted to carry out the acts of the computer-implemented method. For this purpose, the at least one data processing device may store a computer program that includes commands that, when executed by the at least one data processing device, cause the at least one data processing device to carry out the computer-implemented method. The computer-implemented method may also be implemented partly or completely in hardware. The expressions “data processing system” and “at least one data processing device” may be used interchangeably here and below. This also applies to corresponding expressions derived therefrom.

For the case in which the at least one data processing device includes two or more data processing devices, specific acts carried out by the least one data processing device may also be understood as various data processing devices carrying out various acts or various parts of an act. For example, it is not necessary for each data processing device to carry out the acts (e.g., steps). In other words, the carrying out of the acts may be distributed to the two or more data processing devices.

Each form of embodiment of the computer-implemented method produces a corresponding form of embodiment for image display in a limited-angle tomography method, which is not purely computer-implemented, in that corresponding acts for creation of the plurality of projection images (e.g., by an imaging device) are employed.

Receiving or obtaining data or information may, for example, include receiving or obtaining data (e.g., by the data processing system) from a sending entity, reading out of the data from a data memory, or receiving or obtaining a data stream that contains the data, or an extraction of the data from the data stream and so forth. Wired or wireless data transmission may be used to do this. For example, the data may be transmitted between a hardware and/or software interface of the sending entity and a hardware and/or software interface of the data processing system.

The plurality of projection images involves a plurality of x-ray projection images, for example. The method of the present embodiments is, however, principally able to be employed for other imaging modalities that are based on the creation of projection images from various projection directions and on a three-dimensional image reconstruction based thereon.

The plurality of projection images is created according to a limited-angle data acquisition. In other words, the plurality of different projection directions does not cover a complete angular range that would be required for exact three-dimensional reconstruction, but merely a part of the range. For a complete reconstruction, for example, an angular range of at least 180° or of 180° plus the fan angle of the x-ray system is required. In limited-angle tomography, the angular range covered is smaller than 180° (e.g., smaller than 120° or smaller than 60°). For example, the entire angular range covered lies in the range of [30°, 150°], in the range of [30°, 90°], or in the range of [30°, 60°]. The angular range covered relates in this case to a predetermined pivot axis, about which the corresponding x-ray source and where necessary also the x-ray detector are rotated in order to realize the different projection directions. In such cases, both circular trajectories are possible (e.g., pure rotational movements), and also spiral-shaped trajectories, in which, in addition to the rotational movement, at the same time or offset, there is a translational movement along the pivot axis.

The number of the plurality of projection images in limited-angle tomography methods is likewise as a rule far smaller than with CT or CBCT methods and lies, for example, in the range of 30 to 150 images or in the range of 30 to 80 images.

The three-dimensional image reconstruction may be created in accordance with a known method of reconstruction for limited-angle tomography. The three-dimensional image reconstruction is produced by a three-dimensional voxel grid, where a corresponding attenuation value is computed by the reconstruction method for each voxel of the voxel grid based on the plurality of projection images. The voxels of the voxel grid may be cubes or cuboid in shape, but may in some forms of embodiment also have another geometric shape, however.

A projection direction may be understood here and below as a normal direction onto a corresponding projection plane. A sectional plane is to be understood, however, as a sectional plane through the three-dimensional image reconstruction. Accordingly, projection images with a projection direction that is at right angles to the respective sectional plane are not necessarily present for the first sectional plane and the second sectional plane.

A sectional plane is produced by a position and orientation (e.g., in the form of a corresponding normal direction onto the sectional plane). The intersection line especially corresponds to a straight line or a straight section. The position of the intersection line may therefore be understood as a position within the first slice image at right angles to the extent direction of the straight line or the straight section relative to the region of interest. The position of the intersection line may also correspond to a vertical distance of a defined point on the contour of the region of interest from the intersection line.

The first sectional plane and the second sectional plane may be at right angles to one another, but this is not necessarily the case.

The pivot axis will also be referred to below as the z-axis of a cartesian coordinate system. If the object involves a human patient, then the longitudinal axis of the body (e.g., the intersection line of the sagittal plane with the frontal plane of the patient) may be oriented in parallel to the z-axis. This is not an absolute requirement, however.

The projection planes of the plurality of projection images are accordingly especially all parallel to the z-axis, or the z-axis lies within all projection planes. The projection directions of the plurality of projection directions thus lie within the x-y plane of the coordinate system.

The basic principle of limited-angle tomography provides that artifacts due to the angle limitation are the least heavily pronounced in the sectional planes that are parallel or approximately parallel to the central or middle projection direction of the range of angles covered. For example, the angular range may be designated by [α₁,α₂], where an angle of 0° corresponds to the y-axis of the coordinate system and it is true to say that α₁<0°∧α₂>0°. For example, α₁=−α₂. Then, the smallest adverse effect by artifacts for a sectional plane at right angles to the y-axis would be expected, while for sectional planes at right angles to the z-axis or at right angles the x-axis, comparatively strong artifacts would be expected.

The region of interest corresponds to a region in the object that is of particular interest for the respective application or for the user. In such cases, this may involve a specific region in an organ, a lesion, a tumor, or the like.

In order to create a slice image corresponding to a specific sectional plane, which may then be displayed to a user and/or may be used for further processing, a computation region about the sectional plane is defined. The data of the three-dimensional image reconstruction that lies within the computation region (e.g., the data of the corresponding voxels) is then used in order to create the slice image. In this case, for example, the data of the three-dimensional image reconstruction that lies within the computation region may be averaged. In corresponding forms of embodiment, the computation region may also be referred to as the averaging region. As an alternative, it is possible to use data from the plurality of projection images and carry out a new reconstruction of the slice image based on the computation region. The computation region may then be seen as a parameter of the reconstruction itself. The larger the computation region is, the higher the signal-to-noise ratio of the slice image is, for example.

The present embodiments now exploit the fact that, because of low artifact strength in specific sectional planes, a particularly precise and reliable determination of the region of interest is possible. If this is determined based on the first slice image, then the computation region (e.g., the slice thickness and/or position of the computation region) for the creation of the second slice image is adapted to precisely the region of interest (e.g., the position of the intersection line in relation to the region of interest and/or the extent of the region of interest) in order to increase the image quality of the second slice image.

The computation region may correspond to an infinitely extended disk with a constant thickness, which corresponds to the slice thickness of the computation region or to an intersecting set of this disk with the spatial area in which the data of the three-dimensional reconstruction is present. This is not necessarily the case. For example, a wedge-shaped figure may also be provided instead of a disk as the computation region (e.g., thus, a region that is defined by two planes that are not parallel to one another, or another geometric figure). It is also possible for the computation region to include the entire region of interest. The position of the computation region corresponds, for example, to a position along the normal direction of the second sectional plane or a position in relation to the intersection line.

It should be pointed out in this case that a corresponding further computation region may possibly also be used as a basis for creation of the first slice image. This may be predetermined in the conventional way (e.g., thus, have a predetermined slice thickness and a predetermined position in relation to the first sectional plane), so that, for example, the first sectional plane lies in the middle within the further computation region.

The determination of the region of interest includes, for example, the determination of the position of the region of interest in the first slice image and of the geometric extent of the region of interest in the first slice image. For example, a contour of the region of interest in the first slice image may be determined. The region of interest may, for example, be determined by the application of a known segmentation algorithm or using computer aided detection (CAD) or also manually by a user, to whom the first slice image is displayed on a display device.

The geometric extent of the region of interest corresponds, for example, to a geometric extent in a direction at right angles to the intersection line (e.g., to a linear geometric extent in a direction at right angles to the intersection line).

The second sectional plane running through the region of interest may be understood as the second sectional plane at least touching the region of interest (e.g., having at least one point in common with it).

In accordance with at least one form of embodiment, respective projection directions of the plurality of projection directions lie within the x-y plane of a cartesian coordinate system. The plurality of projection directions covers an angular range about a pivot axis that corresponds to a z-axis of the coordinate system. For example, the angular range is symmetrical in relation to the y-axis of the coordinate system.

In accordance with at least one form of embodiment, the plurality of projection directions includes a projection direction that is at right angles to the first sectional plane.

The right-angle alignment in this case includes approximately right-angle alignments (e.g., with a tolerance range of ±δ, where δ=α/N, with the overall number of the plurality of projection directions being N and the angular range covered being α, especially α=|α₁|+|α₂|).

A particular advantage of such forms of embodiment is that the image quality of the first slice image in accordance with such a first sectional plane is especially high or the strength of artifacts in such a first slice image is especially low. For example, the first sectional plane is parallel or approximately parallel to the x-z plane of the coordinate system or, with a corresponding location of the patient, is a frontal plane or coronal plane through the body of the patient.

For example, the projection direction that is at right angles to the first sectional plane is a central or middle projection direction of the angular range covered. For example, the projection directions of the plurality of projection directions is distributed symmetrically about the central or middle projection direction and/or are evenly distributed within the angular range covered.

In accordance with at least one form of embodiment, the second sectional plane is at right angles to the first sectional plane.

This is advantageous since in this case it may be deduced especially precisely from the first slice image whether the second sectional plane runs through the inside of the region of interest or the region of interest touches its contour or where precisely the second sectional plane runs through the region of interest. Accordingly, the position of the intersection line may be determined especially precisely.

In some forms of embodiment, the plurality of different projection directions includes the projection direction that is at right angles to the first sectional plane and in addition is at right angles to the second sectional plane at right angles to the first sectional plane.

This is advantageous since in this case the image quality of the first slice images is especially high and artifacts at right angles to the first sectional plane are especially pronounced.

In accordance with at least one form of embodiment, the second sectional plane is parallel to all projection directions of the plurality of different projection directions.

In such forms of embodiment, the present embodiments have a particularly advantageous effect since artifacts are especially strongly pronounced in such sectional planes. For example, the second sectional plane is parallel to all projection directions of the plurality of different projection directions and in addition the second sectional plane is at right angles to the first sectional plane.

In accordance with at least one form of embodiment, a first boundary position of maximum extent of the region of interest in the direction at right angles to the intersection line and/or a second boundary position of maximum extent of the region of interest in the direction at right angles to the intersection line is determined. The computation region (e.g., the position of the computation region and/or the slice thickness of the computation region) is determined depending on a distance (e.g., a perpendicular distance) of the intersection line from the first boundary position and/or a distance (e.g., a perpendicular distance) of the intersection line from the second boundary position.

In other words, when the first boundary position is determined, but not the second boundary position, the computation region is then determined depending on the distance of the intersection line from the first boundary position. When the second boundary position is determined, but not the first boundary position, the computation region is then determined depending on the distance of the intersection line from the second boundary position. When the first boundary position and the second boundary position are determined, the computation region is then determined depending on the distance of the intersection line from the first boundary position and/or depending on the distance of the intersection line from the second boundary position.

The boundary positions correspond especially to points on the contour of the region of interest in the first slice image between which the region of interest extends in a direction at right angles to the intersection line. If the second sectional plane is at right angles to the first sectional plane, then the intersection line in the first slice image runs especially horizontally. The first boundary position then corresponds, for example, to an upper boundary position in the vertical direction, and the second boundary position corresponds for example to a lower boundary position in the vertical direction.

Taking account of the first boundary position and/or second boundary position for determination of the computation region enables the region always to be chosen so that the second slice image may reproduce the relevant structures in the region of interest especially well. For example, what may be achieved in this way is that the computation region is located completely within the region of interest, provided this is able to be determined in the first slice image.

In accordance with at least one form of embodiment, the position of the computation region is determined depending on the position of the intersection line. For example, in this case, the second sectional plane is at right angles to the first sectional plane.

Thus, the position of the computation region may be defined dynamically in relation to the intersection line depending on the position of the intersection line in the region of interest. For example, the position of the computation region may be chosen such that the intersection line does not lie in the center of the computation region when this is advantageous for the creation of the second slice image (e.g., when what may be achieved by this is that a larger part of the computation region or the whole computation region lies in the region of interest).

For example, the position of the computation region may be determined depending on the distance of the intersection line from the first boundary position and/or the distance of the intersection line from the second boundary position.

In accordance with at least one form of embodiment, the position of the intersection line corresponds to the first boundary position or the second boundary position. The position of the computation region and/or the slice thickness are determined such that a part of the computation region that lies outside the region of interest is smaller than a part of the computation region that lies within the region of interest, or such that the computation region lies completely within the region of interest. In this case, for example, the second sectional plane is at right angles to the first sectional plane.

What is achieved by this is that, for computation of the second slice image, a larger proportion of the region of interest is taken into account and/or a smaller part of the object that lies outside the region of interest than would be the case in the conventional definition of the computation region regardless of the position of the intersection line. The boundary of the region of interest is further obtained by this. The image quality of the second slice image may be increased by this.

In accordance with at least one form of embodiment, the position and the slice thickness of the computation region are determined such that the part of the computation region that lies outside the region of interest, regardless of the position of the intersection line, is smaller than the part of the computation region that lies within the region of interest, or such that the computation region, regardless of the position of the intersection line, lies completely within the region of interest. In this case, for example, the second sectional plane is at right angles to the first sectional plane.

In other words, there is provision that, for any given position of the intersection line in the region of interest (e.g., between first boundary position and second boundary position), where the first boundary position and the second boundary position are included as possible positions of the intersection line, the computation region always lies completely within the region of interest or at least the part of the computation region that lies outside the region of interest is always smaller than the remaining part of the computation region.

What is achieved by this is that, for computation of the second slice image, a larger proportion of the region of interest is taken into account and/or a smaller part of the object that lies outside the region of interest than would be the case for conventional definition of the computation region, regardless of the position of the intersection line. The image quality of the second slice image may be further increased by this.

In accordance with at least one form of embodiment, the slice thickness of the computation region is determined depending on the position of the intersection line. In this case, for example, the second sectional plane is at right angles to the first sectional plane.

Thus, the slice thickness of the computation region may be defined dynamically depending on the position of the intersection line in the region of interest. The greater the slice thickness is, the better, for example, the image quality of the second slice image may be. The determination of the slice thickness thus enables the region available to be better utilized, without a very large part of the computation region having to lie outside the region of interest.

For example, the slice thickness of the computation region may be determined depending on the distance of the intersection line from the first boundary position and/or the distance of the intersection line from the second boundary position.

In accordance with at least one form of embodiment, the slice thickness of the computation region is determined depending on the geometric extent of the region of interest in the first sectional plane (e.g., depending on a distance of the first boundary position to the second boundary position in a direction at right angles to the intersection line).

Thus, the slice thickness of the computation region may be defined dynamically depending on the geometric extent of the region of interest. The larger the slice thickness is, the better, for example, the image quality of the second slice image may be.

In accordance with at least one form of embodiment, data of the plurality of projection images that corresponds to the computation region is extracted, and the second slice image is created based on the extracted data.

In other words, in such forms of embodiment, the second slice image for the second sectional plane is created in accordance with the computation region based directly on the plurality of projection images.

The creation of the second slice image may, for example, correspond to a new reconstruction (e.g., to a new three-dimensional image reconstruction), or to a part reconstruction based on the computation region. This may also include averaging steps of the corresponding data within the computation region. For example, a dynamic average value formation may be used, whereby the boundaries of the region of interest are retained.

An advantage of creation of the second slice image based directly on the plurality of projection images is that the plurality of projection images are present in a higher spatial resolution than the three-dimensional image reconstruction. The image quality of the second slice image may thus be further improved.

In accordance with at least one form of embodiment, data is extracted of the image reconstruction that corresponds to the computation region, and the second slice image is created based on the data extracted in this way.

In other words, in such forms of embodiment, the second slice image is created indirectly based on the plurality of projection images, since the three-dimensional image reconstruction has likewise been created depending on the plurality of projection images.

The creation of the second slice image may include an averaging of data of the image reconstruction within the computation region. For example, a dynamic average value formation may be used, whereby the boundaries of the region of interest are retained. An advantage of creation of the second slice image based on the data of the image reconstruction is that this data is already available in any case, and the computation effort for creation of the second slice image may be reduced by this.

In accordance with a further aspect of the present embodiments, a data processing system that is configured to carry out a computer-implemented method of the present embodiments is provided.

In the present disclosure, the expressions “data processing system” and “at least one data processing device” may be used interchangeably. A data processing device may be understood as data processing device that contains a processing circuit. The data processing device may thus especially process data for carrying out computing operations. This might possibly also include operations for carrying out indexed accesses to a data structure (e.g., a look-up table (LUT)) as well as a data processing process able to be implemented in hardware.

The data processing device may contain one or more computers, one or more microcontrollers, and/or one or more integrated circuits (e.g., one or application-specific integrated circuits (ASIC)), one or more Field Programmable Gate Arrays (FPGA), and/or one or more Systems on a Chip (SoC). The data processing device may also include one or more processors (e.g., one or more microprocessors, one or more Central Processing Units (CPU), one or more Graphics Processing Units (GPU), and/or one or more signal processors, such as one or more Digital Signal Processors (DSP)). The data processing device may also include a physical or a virtual network of computers or of other of the units.

In various forms of embodiment, the data processing device includes one or more hardware and/or software interfaces and/or one or more memory units.

A memory unit may be embodied as volatile data memory (e.g., as a Dynamic Random Access Memory (DRAM) or Static Random Access Memory (SRAM)), or as a non-volatile data memory (e.g., as Read-Only Memory (ROM), as Programmable Read-Only Memory (PROM), as Erasable Programmable Read-Only Memory (EPROM), as Electrically Erasable Programmable Read-Only Memory (EEPROM), as flash memory or Flash EEPROM, as Ferroelectric Random Access Memory (FRAM), as Magnetoresistive Random Access Memory (MRAM), or as Phase-Change Random Access Memory (PCRAM)).

In accordance with a further aspect of the present embodiments, an imaging system for limited-angle tomography is specified. The imaging system has a data processing system of the present embodiments and also an imaging device that is configured to create the plurality of projection images.

In accordance with at least one form of embodiment of the imaging system, the imaging device involves an x-ray imaging device (e.g., a C-arm device).

In accordance with at least one form of embodiment, the imaging system has a display device. The data processing system is configured to display the second slice image on the display device.

In some forms of embodiment, the display device may also be regarded as part of the data processing system.

Further forms of embodiment of the imaging system follow on directly from the various embodiments of the computer-implemented method of the present embodiments and vice versa. For example, individual features and corresponding explanations, as well as advantages with regard to the various forms of embodiment for the computer-implemented method, may be transferred by analogy to corresponding forms of embodiment of the imaging system. For example, the imaging system of the present embodiments is embodied or programmed for carrying out a computer-implemented method of the present embodiments. For example, the imaging system of the present embodiments carries out the computer-implemented method of the present embodiments.

In accordance with a further aspect of the present embodiments, a computer program with commands is specified. When the commands are executed by a data processing system, the commands cause the data processing system to carry out a computer-implemented method of the present embodiments.

The commands may be present, for example, as program code. The program code may, for example, be provided as binary code or Assembler and/or as source code of a programming language (e.g., C) and/or as a program script (e.g., Python).

In accordance with a further aspect of the present embodiments, a computer-readable memory medium (e.g., a non-transitory computer-readable storage medium) is specified (e.g., a physical and/or non-volatile computer-readable memory medium that stores a computer program of the present embodiments).

The computer program and the computer-readable memory medium are computer program products with the commands in each case.

Further features and combinations of features of the invention emerge from the figures and from their description, as well as from the claims. For example, further forms of embodiment of the invention do not absolutely have to contain all features of one of the claims. Further forms of embodiment of the inventions may have features or combinations of features that are not given in the claims.

The invention will be explained in greater detail below with the aid of specific example embodiments and associated schematic drawings. In the figures, elements that are the same or have the same functions may be provided with the same reference numbers. The description of elements that are the same or have the same functions may possibly not necessarily be repeated with regard to different figures.

### BRIEF DESCRIPTION OF THE DRAWINGS

FIG. 1 shows a schematic diagram of an example of a form of embodiment of an imaging system for limited-angle tomography;

FIG. 2 shows a schematic block diagram of an example of a form of embodiment of a computer-implemented method for image display;

FIG. 3 shows a schematic diagram of first slice images and computation regions for second slice images in accordance with a further example of a form of embodiment of a computer-implemented method for image display;

FIG. 4 shows a schematic diagram of first slice images and computation regions for second slice images in accordance with a further example of a form of embodiment of a computer-implemented method for image display; and

FIG. 5 shows a schematic diagram of first slice images and computation regions for second slice images in accordance with a further example of a form of embodiment of a computer-implemented method for image display.

### DETAILED DESCRIPTION

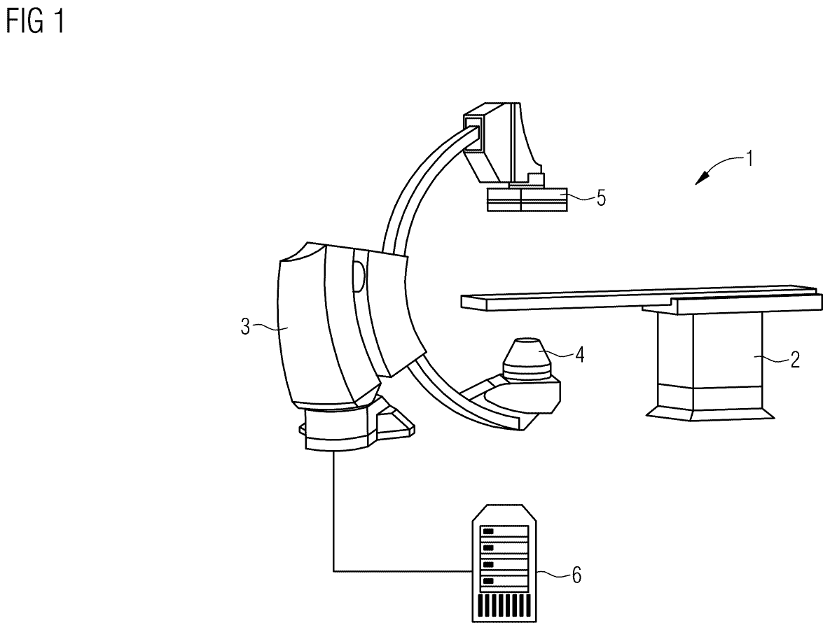

Shown schematically in FIG. 1 is an example of a form of embodiment of an imaging system 1 for limited-angle tomography.

The imaging system 1 has an imaging device 3 that is configured to create a plurality of projection images that represent an object 8 and correspond to a plurality of different projection directions.

The object 8 may, for example, be a patient or a part of the patient's body. The patient may be placed on a patient couch 2 of the imaging system 1.

By way of example, the imaging device 3 is shown as a C-arm x-ray device with an x-ray source 4 and an x-ray detector 5. The explanations given below, however, are able to be transferred by analogy to other imaging devices 3 that may create projection images that show an object 8 from a plurality of different projection directions. The plurality of different projection directions is realized, for example, by the x-ray source 4 and the x-ray detector 5 being rotated on a trajectory (e.g., a circular or spiral trajectory) about a pivot axis, so that a predetermined angular range is covered. The pivot axis in this case is, for example, parallel to or the same as the longitudinal axis of the patient's body.

The imaging system 1 has an embodiment of a data processing system 6 that is configured, based on the plurality of projection images, to carry out a computer-implemented method of the present embodiments for image display in a limited-angle tomography method.

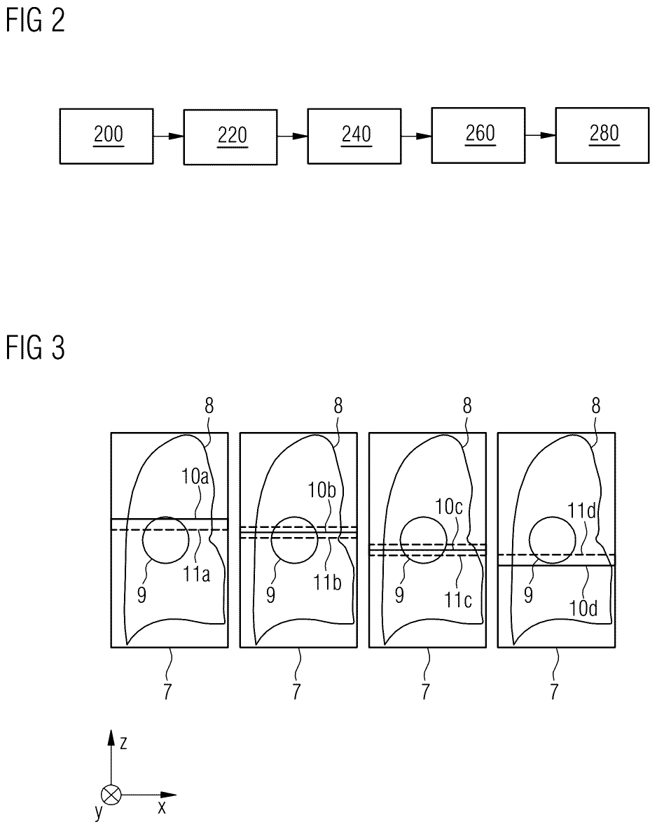

FIG. 2 shows a schematic block diagram of an example of a form of embodiment of such an embodiment of a computer-implemented method. Further aspects of various forms of embodiment of the computer-implemented method are shown in FIGS. 3 to 5.

In this method, in act 200, the plurality of projection images is obtained, and based on the plurality of projection images, a three-dimensional image reconstruction is created. In act 220, a first slice image 7 for a first sectional plane through the object 8 based on the image reconstruction is created, and a region of interest 9 of the object 8 in the first slice image 7 is determined.

In the examples of FIGS. 3 to 5, the first sectional plane is, for example, a coronal plane of the patient through the object 8 (e.g., the lungs of the patient). The region of interest 9 may, for example, correspond to a tumor or to another lesion.

In act 240, in the first sectional plane, an intersection line 10, 10a, 10b, 10c, 10d of a second sectional plane, which runs through the region of interest 9, is determined. The second sectional plane may, for example, be at right angles to the first sectional plane x-z. In the example of FIGS. 3 to 5, the second sectional plane x-y corresponds, for example, to a transversal plane or axial plane through the patient's body.

In act 260, depending on a position of the intersection line 10, 10a, 10b, 10c, 10d and/or depending on a geometric extent of the region of interest 9 in the first slice image 7, a computation region 11, 11a, 11b, 11c, 11d about the second sectional plane is determined. To do this, a slice thickness of the computation region 11, 11a, 11b, 11c, 11d and a position of the computation region 11, 11a, 11b, 11c, 11d in relation to the intersection line 10, 10a, 10b, 10c, 10d may be determined.

In act 280, based on the plurality of projection images, a second slice image for the second sectional plane is created in accordance with the computation region 11, 11a, 11b, 11c, 11d. This may be undertaken, for example, based directly on the plurality of projection images or based on the three-dimensional image reconstruction (e.g., based indirectly on the plurality of projection images).

Shown in the example of FIG. 3, from left to right, are first sectional images 7 with four different positions of the intersection line 10a, 10b, 10c, 10d. The slice thickness of the computation region 11a, 11b, 11c, 11d in this case is the same for all four cases, and the position of the computation region 11a, 11b, 11c, 11d is defined depending on the position of the intersection line 10a, 10b, 10c, 10d (e.g., such that the computation region 11a, 11b, 11c, 11d in all four cases lies completely within the region of interest 9, where in this sense, the contour of the region of interest 9 also lies within the region of interest 9).

In the first sectional image 7 on the far left, the position of the intersection line 10a corresponds to an upper boundary position of the region of interest 9. The computation region 11a extends out from the intersection line 10a exclusively downward into the region of interest 9. Upward and downward in this case refer to the positive or negative z direction. In the first sectional image 7 at the second position from the right the intersection line 10b is located below the upper boundary position of the region of interest 9. The computation region 11b extends out from the intersection line 10b symmetrically upward and downward. The same applies by analogy for the first sectional image 7 at the third position from the left, the intersection line 10c, and the computation region 11c. In first sectional image 7 on the far right, the position of the intersection line 10d corresponds to a lower boundary position of the region of interest 9. The computation region 11d extends out from the intersection line 10a exclusively upward into the region of interest 9.

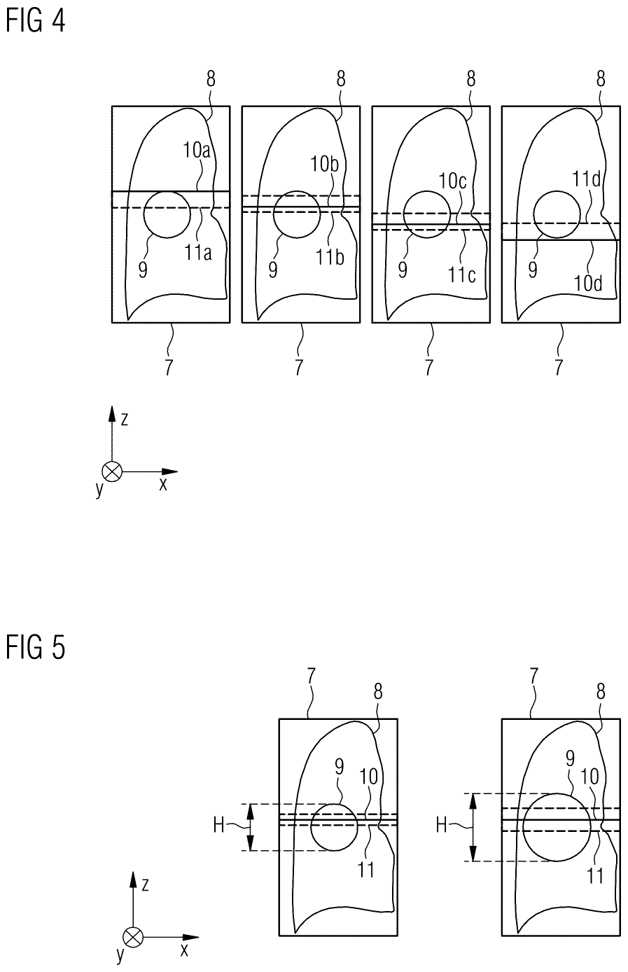

As in FIG. 3, shown in the example of FIG. 4 from left to right are first sectional images 7 with four different positions of the intersection line 10a, 10b, 10c, 10d. The positions of the intersection line 10a, 10b, 10c, 10d correspond to those shown in FIG. 3. The slice thickness of the computation region 11a, 11b, 11c, 11d in this case is, however, likewise dependent on the positions of the intersection lines 10a, 10b, 10c, 10d. This enables different parts of the region of interest 9 corresponding to the specific requirements for the second slice image to be used.

Shown in FIG. 5 are two first sectional images 7 with different geometric extents of the region of interest 9 (e.g., the extent H between upper boundary position and lower boundary position in the z direction differs). In the case shown above, H is smaller than in the case shown below. Here, the slice thickness of the computation region 11 is defined depending on the geometric extent of the region of interest 9. For the case with the greater extent H, the slice thickness of the computation region 11 is also especially greater.

As described, the present embodiments make possible an image display in limited-angle tomography with enhanced image quality.

In some forms of embodiment, an automatic adaptation of the slice thickness of the computation region 11a, 11b, 11c, 11d for an axial slice image based on the region of interest 9, which is to be seen on a coronal slice image, is provided. The region of interest 9 corresponds for example to a lesion. The difference in the ability of the lesion to be detected in various anatomical planes may be exploited by this. Because of the good detectability of the lesion in the sagittal plane, it may, for example, be detected using a CAD algorithm. The CAD algorithm delivers the location of the lesion in the sagittal plane, from which the slice thickness for the axial plane may be determined. As an alternative, the lesion may also be marked manually. Assuming that the cranial-caudal axis is referred to as the z-axis (e.g., the axis at right angles to the axial slice), the slice thickness may be adapted based on the z position of the intersection line 10a, 10b, 10c, 10d within the region of interest 9.

Further advantages of various example embodiments include simple integration into existing reconstruction and visualization methods without changing the image data, which would increase the risk for information loss or artifacts that are not present in the raw data. Further, no additional imaging data is required, such as, for example, from prior imaging.

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description

1. A computer-implemented method for image display in a limited-angle tomography method, the computer-implemented method comprising:
obtaining a plurality of projection images that represent an object and correspond to a plurality of different projection directions, and creating, based on the plurality of projection images, a three-dimensional image reconstruction;
creating a first slice image for a first sectional plane through the object based on the three-dimensional image reconstruction, and determining a region of interest of the object in the first slice image;
determining, in the first sectional plane, an intersection line of a second sectional plane that runs through the region of interest;
determining, depending on a position of the intersection line, depending on a geometric extent of the region of interest in the first slice image, or depending on a combination thereof, a computation region around the second sectional plane, wherein the computation region has a position and a slice thickness; and
creating, based on the plurality of projection images, a second slice image for the second sectional plane in accordance with the computation region.

2. The computer-implemented method of claim 1, wherein the second sectional plane is at right angles to the first sectional plane.

3. The computer-implemented method of claim 2, wherein:
a first boundary position of maximum extent of the region of interest in a direction at right angles to the intersection line, a second boundary position of maximum extent of the region of interest in a direction at right angles to the intersection line, or a combination thereof is determined; and
the computation region is determined depending on a distance of the intersection line from the first boundary position, a distance of the intersection line from the second boundary position, or a combination thereof.

4. The computer-implemented method of claim 3, further comprising determining a position of the computation region depending on the position of the intersection line.

5. The computer-implemented method of claim 4, wherein the position of the intersection line corresponds to the first boundary position or to the second boundary position, and the position of the computation region is determined such that a part of the computation region that lies outside the region of interest is smaller than a part of the computation region that lies within the region of interest, or such that the computation region lies completely within the region of interest.

6. The computer-implemented method of claim 1, wherein the position and the slice thickness of the computation region are determined such that:
a part of the computation region that lies outside the region of interest, independent of the position of the intersection line, is smaller than a part of the computation region that lies within the region of interest; or
the computation region, independent of the position of the intersection line, lies completely within the region of interest.

7. The computer-implemented method of claim 1, wherein the slice thickness of the computation region is determined depending on the position of the intersection line.

8. The computer-implemented method of claim 1, wherein the slice thickness of the computation region is determined depending on the geometric extent of the region of interest in the first sectional plane.

9. The computer-implemented method of claim 1, wherein data of the plurality of projection images that corresponds to the computation region is extracted, and the second slice image is created based on the extracted data.

10. The computer-implemented method of claim 1, wherein data of the image reconstruction, which corresponds to the computation region is extracted, and the second slice image is created based on the extracted data.

11. The computer-implemented method of claim 1, wherein the plurality of different projection directions includes a projection direction that is at right angles to the first sectional plane, the second sectional plane is parallel to all projection directions of the plurality of different projection directions, or a combination thereof.

12. A data processing system comprising:
a processor configured for image display in a limited-angle tomography method, the processor being configured for image display in the limited-angle tomography method comprising the processor being configured to:
obtain a plurality of projection images that represent an object and correspond to a plurality of different projection directions, and create, based on the plurality of projection images, a three-dimensional image reconstruction;
create a first slice image for a first sectional plane through the object based on the three-dimensional image reconstruction, and determine a region of interest of the object in the first slice image;
determine, in the first sectional plane, an intersection line of a second sectional plane that runs through the region of interest;
determine, depending on a position of the intersection line, depending on a geometric extent of the region of interest in the first slice image, or depending on a combination thereof, a computation region around the second sectional plane, wherein the computation region has a position and a slice thickness; and
create, based on the plurality of projection images, a second slice image for the second sectional plane in accordance with the computation region.

13. An imaging system for limited-angle tomography, the imaging system comprising: a data processing system comprising:
a processor configured for image display in a limited-angle tomography method, the processor being configured for image display in the limited-angle tomography method comprising the processor being configured to:
obtain a plurality of projection images that represent an object and correspond to a plurality of different projection directions, and create, based on the plurality of projection images, a three-dimensional image reconstruction;
create a first slice image for a first sectional plane through the object based on the three-dimensional image reconstruction, and determine a region of interest of the object in the first slice image;
determine, in the first sectional plane, an intersection line of a second sectional plane that runs through the region of interest;
determine, depending on a position of the intersection line, depending on a geometric extent of the region of interest in the first slice image, or depending on a combination thereof, a computation region around the second sectional plane, wherein the computation region has a position and a slice thickness; and
create, based on the plurality of projection images, a second slice image for the second sectional plane in accordance with the computation region; and
an imaging device configured to create the plurality of projection images.

14. The imaging system of claim 13, wherein the imaging device is configured as an x-ray C-arm device.
